(19) United States
(12) Patent Application Publication
(10) Pub. No.: US 2004/0180879 A1
(43) Pub. Date: Sep. 16, 2004

(54) Novel method of treating vasomotor symptoms

(76) Inventors: Darlene Coleman Deecher, Quakertown, PA (US); Istvan Jozsef Merchenthaler, Chester Springs, PA (US)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 10/685,974

(22) Filed: Oct. 14, 2003

Related U.S. Application Data
(60) Provisional application No. 60/418,516, filed on Oct. 15, 2002.

(51) Int. Cl. A61K 31/5415; A61K 31/445

(52) U.S. Cl. 514/225.8; 514/317

(57) ABSTRACT

The present invention relates to the use of compounds and composition of compounds that modulate norepinephrine levels for the treatment of vasomotor symptoms such as thermoregulatory disorders. Furthermore, the present invention relates to the use of compounds and compositions of compounds having norepinephrine reuptake inhibitor (NRI) activity alone or norepinephrine reuptake inhibitor and serotonin reuptake inhibitor (NRI/SRI) dual activity in combination with 5-HT₂ₐ receptor antagonist activity.

### CROSS-REFERENCE TO RELATED APPLICATIONS

This application claims benefit of U.S. application Ser. No. 60/418,516, filed Oct. 15, 2002, the disclosure of which is incorporated herein by reference in its entirety.

### FIELD OF THE INVENTION

The present invention relates to the use of compounds and composition of compounds that modulate norepinephrine (NE) levels in combination with 5-HT₂ₐ receptor antagonist for the treatment of vasomotor symptoms such as hot flush. The combined NE modulating activity with a 5-HT₂ₐ receptor antagonist activity may reside within the same compound or in two or more different compounds.

### BACKGROUND OF THE INVENTION

Vasomotor symptoms (VMS), referred to as hot flushes and night sweats, are the most common symptoms associated with menopause, occurring in 60% to 80% of all women following natural or surgically-induced menopause. VMS are likely to be an adaptive response of the central nervous system (CNS) to declining sex steroids. To date, the most effective therapies for VMS are hormone-based treatments, including estrogens and/or some progestins. Hormonal treatments are very effective at alleviating VMS, but they are not appropriate for all women.

It is well recognized that VMS are caused by fluctuations of sex steroid levels and can be disruptive and disabling in both males and females. A hot flush can last up to thirty minutes and vary in their frequency from several times a week to multiple occurrences per day. The patient experiences a hot flash as a sudden feeling of heat that spreads quickly from the face to the chest and back and then over the rest of the body. These are usually accompanied by outbreaks of profuse sweating. They sometimes occur several times an hour, and they often occur at night. Hot flushes and outbreaks of sweats occurring during the night can cause sleep deprivation. Psychological and emotional symptoms observed such as nervousness, fatigue, irritability, insomnia, depression, memory loss, headache, anxiety, nervousness or inability to concentrate are considered to be caused by the sleep deprivation following hot flush and night sweats (Kramer et al., In: Murphy et al., 3^{rd} Int'l Symposium on Recent Advances in Urological Cancer Diagnosis and Treatment-Proceedings. Paris, France: SCI: 3-7 (1992)).

Hot flushes may be even more severe in women treated for breast cancer for several reasons: 1) many survivors of breast cancer are given tamoxifen, the most prevalent side effect of which is hot flush, 2) many women treated for breast cancer undergo premature menopause from chemotherapy, 3) women with a history of breast cancer have generally been denied estrogen therapy because of concerns about potential recurrence of breast cancer (Loprinzi, C. L., et al Lancet, 2000, 356(9247): p. 2059-2063).

Men also experience hot flushes following steroid hormone (androgen) withdrawal. This is true in cases of age-associated androgen decline (Katovich et al., Proceedings of the Society for Experimental Biology & Medicine, 1990. 193(2): p. 129-35) as well as in extreme cases of hormone deprivation associated with treatments for prostate cancer (Berendsen et al, European Journal of Pharmacology, 2001. 419(1): p. 47-54). As many as one-third of these patients will experience persistent and frequent symptoms severe enough to cause significant discomfort and inconvenience.

The precise mechanism of these symptoms is unknown but generally is thought to represent disturbances to normal homeostatic mechanisms controlling thermoregulation and vasomotor activity (Kronenberg et al., “Thermoregulatory Physiology of Menopausal Hot Flashes: A Review,” Can. J. Physiol. Pharmacol., 65:1312-1324 (1987)).

The fact that estrogen treatment (e.g. estrogen replacement therapy) relieves the symptoms establishes the link between these symptoms and an estrogen deficiency. For example, the menopausal stage of life is associated with a wide range of other acute symptoms as described above and these symptoms are generally estrogen responsive.

It has been suggested that estrogens may stimulate the activity of both the norepinephrine (NE) and/or serotonin (5-HT) systems (J. Pharmacology & Experimental Therapeutics, 1986, 236(3): 646-652).

Although VMS are most commonly treated by hormone therapy (orally, transdermally, or via an implant), some patients may not tolerate estrogen treatment (Berendsen, Maturitas, 2000. 36(3): p. 155-164, Fink et al., Nature, 1996. 383(6598): p. 306). In addition, hormone replacement therapy is usually not recommended for women or men with or at risk for hormonally sensitive cancers (e.g. breast or prostate cancer). Thus, non-hormonal therapies (e.g. fluoxetine, paroxetine [SRIs] and clonidine) are being evaluated clinically. WO9944601 discloses a method for decreasing hot flushes in a human female by administering fluoxetine. Other options have been studied for the treatment of hot flashes, including steroids, alpha-adrenergic agonists, and beta- blockers, with varying degree of success (Waldinger et al., Maturitas, 2000. 36(3): p.165-168).

Given the complex multifaceted nature of thermoregulation and the interplay between the CNS and PNS in maintaining thermoregulatory homeostatsis, multiple therapies and approaches can be developed to target vasomotor symptoms. The present invention focuses on novel methods of modulating the noradrenergic system in combination with 5-HT₂ₐ receptor system to alleviate vasomotor symptoms.

### SUMMARY OF THE INVENTION

The present invention provides a method for treating a subject afflicted with vasomotor symptoms comprising administering to said subject a therapeutically effective amount of one or more compounds having norepinephrine reuptake inhibitor (NRI) activity, and 5-HT₂ₐ receptor antagonist activity. The invention further provides a method wherein the norepinephrine reuptake inhibitor (NRI) activity and 5-HT₂ₐ receptor antagonist activity are provided by a single compound or by two or more compounds and their derivatives thereof. The compounds of present invention may be administered with one or more pharmaceutically acceptable salts.

The present invention provides a method for treating a subject afflicted with vasomotor symptoms comprising administering to said subject a therapeutically effective amount of one or more compounds having NRI/SRI activity, and 5-HT₂ₐ antagonist activity. NRI/SRI activity and 5-HT₂ₐ receptor antagonist activities may be provided by a single compound or two or more compounds. The compounds of present invention may be administered with one or more pharmaceutically acceptable salts.

The present invention also provides a method where administration of norepinephrine reuptake inhibitor and 5-HT₂ₐ receptor antagonist is concurrent or simultaneous.

### BRIEF DESCRIPTION OF THE DRAWINGS

The invention can be more fully understood from the following detailed description and accompanying drawings that form a part of this application.

FIG. 1 shows the ability of a 5-HT₂ₐ receptor agonist (DOI; 0.3 mg/kg) to block a 5-HT₂ₐ receptor antagonist (MDL-100907; 0.01 and 0.1 mg/kg) -induced flush in a morphine-dependent rat model (MD model) of hot flush compared to vehicle control. FIG. 1 also demonstrates the ability of the 5-HT₂ₐ receptor antagonist to reverse the efficacy of the 5-HT₂ₐ agonist in abating the naloxone-induced flush. Thus, the present data suggests that the 5-HT₂ₐ receptor is involved in thermoregulation in this MD model. * indicates p<0.05 () (referred to in Example 1).

FIG. 2 demonstrates the effect of a known NRI (desipramine; 1 mg/kg) in combination with a 5-HT₂ₐ receptor antagonist (MDL-100907; 0.01 mg/kg) in a MD model of hot flush. * indicates p<0.05 compared to vehicle control. ψ indicates p<0.05 compared to MDL-100907. Φ indicates p<0.05 compared to desipramine. FIG. 2A shows 5-HT₂ₐ receptor-induced flush- 15 min pre-naloxone; desipramine abates an MDL-100907-induced flush. FIG. 2B shows opioid receptor involvement in a naloxone-induced flush- 15 min post naloxone; MDL-100902 enhances desipramine's effect on abating naloxone-induced flush (referred to in Example 2).

FIG. 3 shows the effect of venlafaxine (10 mg/kg) in combination with MDL-100907 (5-HT₂ₐ receptor antagonist; 0.01 mg/kg) in the MD model. FIG. 3A shows 5-HT₂ₐ receptor-mediated induced flush-15 min pre-naloxone; venlafaxine abates an MDL-100907-induced flush. FIG. 3B shows opioid receptor-mediated naloxone-flush-15 min post naloxone; MDL-100902 enhances venlafaxine's effect on a naloxone-induced flush* indicates p<0.05 compared to vehicle control. ψ indicates p<0.05 compared to MDL-100907 (referred to in Example 3).

### DETAILED DESCRIPTION OF THE INVENTION

The present invention provides methods for treating a subject afflicted with vasomotor symptoms comprising administering NRIs or dual acting NRI/SRIs in combination with at least one 5-HT₂ₐ receptor antagonist. The invention also includes pharmaceutical compositions and products containing NRIs or dual acting NRI/SRIs with 5-HT₂ₐ receptor antagonists.

It is believed that the present invention described presents a substantial breakthrough in the field of treatment, alleviation, inhibition, and/or prevention of vasomotor symptoms such as vasomotor instability. It was discovered that using NRI compounds or dual acting NRI/SRI compounds in combination with a 5-HT₂ₐ receptor antagonist surprisingly results in such benefits as clearer dose-related definitions of efficacy, diminished reported side effect, superior therapy due to enhanced activity, and accordingly, an improved therapeutic index. For example, high doses of NRIs (e.g. 300-500 mg/day; desipramine) or NRI/SRI (e.g. 20 mg/day; fluoxetine) alone can induce vomiting, nausea, sweating and flushes (Janowsky, et al., Journal of Clinical Psychiatry, 1984. 45(10 Pt 2): p. 3-9). The present invention provides treatment or prevention of vasomotor symptoms while diminishing side effects caused by using higher doses of NRI or NRI/SRIs alone.

In one embodiment, a 5-HT₂ₐ receptor antagonist when administered alone induced a rapid rise in tail skin temperature (TST) of the rat model of vasomotor instability and that this effect was a 5-HT₂ₐ receptor mediated event described herein. In particular, a 5-HT₂ₐ receptor antagonist such as MDL-100907 induced hot flush and that the flush was abated in the presence of a 5-HT₂ₐ receptor agonist, DOI.

In another embodiment, it was found that the noradrenergic and the 5-HT₂ₐ receptor systems play an important role in maintaining normal body temperature. A known NRI compound, desipramine (approximate ED₅₀), was able to abate 50% of naloxone-induced hot flush in animal models of vasomotor instability. In addition, desipramine administered after the 5-HT₂ₐ receptor antagonist abated the 5-HT₂ₐ receptor-mediated pre-flush by 75%. Surprisingly, desipramine when co-administered with a 5-HT₂ₐ receptor antagonist resulted in significantly enhanced (80%) abatement of a naloxone-induced flush. Therefore, co-administration of NRI and 5-HT₂ₐ receptor antagonist compound was more efficacious for abating a naloxone-induced flush.

Examples of NRI include but are not limited to norpramine, desipramine, nisoxetine, reboxetine, imipramine, maprotiline amoxapine, doxepin, lofepramin, and amitriptyline, and pharmaceutically acceptable thereof.

In yet another embodiment, when compounds with dual NRI/SRI activity (NRI/SRI compounds) such as venlafaxine were administered after the 5-HT₂ₐ receptor antagonist such as (+)-a-(2,3-dimethoxyphenyl)-1-[2-(4-fluorophenyl)ethyl]-4-piperidinemethanol (MDL-100907), the 5-HT₂ₐ receptor-induced flush in a rat was abated by 60% when compared to MDL treated animals. Additionally, venlafaxine when co-administered with a 5-HT₂ₐ receptor antagonist showed enhanced abatement of a naloxone-induced flush.

Examples of dual acting NRI/SRI compounds are venlafaxine, desvenlafaxine (DVS-233), milnacipran, and duloxetine, and pharmaceutically acceptable salt thereof. In the case of dual acting NRI/SRI compounds, the compound may exhibit more NRI activity, more SRI, or approximately equivalent NRI and SRI activity.

Examples of 5-HT₂ₐ receptor antagonist include compounds of the formula:

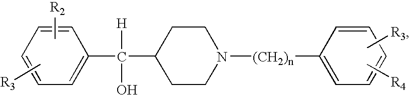

such as those disclosed in European Application EP-A-0,208,235, and (+)-α-(2,3-dimethoxyphenyl)-1-[2-(4-fluorophenyl)ethyl]-4-piperidinemethanol and pharmaceutically acceptable salts thereof, disclosed in WO 91/18602 (available under the trade name MDL-100907), the disclosures of which are incorporated by reference, in their entireties.

Accordingly, any combination of the above mentioned NRIs or NRI/SRIs with 5-HT₂ₐ receptor antagonists may be used to maintain normal body temperature with the added advantage of reducing side effects noted for higher doses of NRIs or dual acting NRI/SRIs such as sweating.

Definition of Abbreviations and Terms:

The following definitions are provided for the full understanding of terms and abbreviations used in this specification.

As used herein and in the appended claims, the singular forms “a”, “an”, and “the” include the plural reference unless the context clearly indicates otherwise. Thus, for example, a reference to “an antagonist” includes a plurality of such antagonists, and a reference to “a compound” is a reference to one or more compounds and equivalents thereof known to those skilled in the art, and so forth.

The abbreviations in the specification correspond to units of measure, techniques, properties or compounds as follows: “min” means minutes, “h” means hour(s), “μL” means microliter(s), “mL” means milliliter(s), “mM” means millimolar, “M” means molar, “mmole” means millimole(s), “cm” means centimeters, “SEM” means standard error of the mean and “IU” means International Units. “Δ° C.” and ΔTST mean change in tail skin temperature normalized for 15 min baseline TST prior to naloxone-induced flush. “ED₅₀ value” means dose which results in 50% alleviation of flush (50% mean maximum endpoint).

“Tail skin temperature” is abbreviated TST.

“Norepinephrine reuptake inhibitor” is abbreviated NRI.

“Norepinephrine transporter” is abbreviated NET.

“Selective norepinephrine reuptake inhibitor” is abbreviated SNRI.

“Serotonin reuptake inhibitor” is abbreviated SRI.

“Selective serotonin reuptake inhibitor” is abbreviated SSRI.

“Norepinephrine” is abbreviated NE.

“Serotonin is abbreviated 5-HT.

“Serotonin 2a receptor is abbreviated 5-HT₂ₐ.

“Subcutaneous” is abbreviated sc.

In the context of this disclosure, a number of terms shall be utilized. The term “treatment” as used herein includes preventative (e.g., prophylactic), curative or palliative treatment and “treating” as used herein also includes preventative, curative and pallative treatment.

A “therapeutically effective amount” refers to an amount effective, at dosages and for periods of time necessary, to achieve the desired result. Desired result may include treating, preventing, alleviating or inhibiting vasomotor symptoms or its related thermoregulatory disorders. Varying hormone levels will influence the amount of compound required in the present invention. For example, the perimenopausal state may require lower doses of a compound due to higher hormone levels than the menopausal state.

It will be appreciated that the therapeutically effective amount of components of the present invention will vary from patient to patient not only with the particular compound, component or composition selected, the route of administration, and the ability of the components (alone or in combination with one or more combination drugs) to elicit a desired response in the individual, but also with factors such as the disease state or severity of the condition to be alleviated, hormone levels, age, sex, weight of the individual, the state of being of the patient, and the severity of the pathological condition being treated, concurrent medication or special diets then being followed by the particular patient, and other factors which those skilled in the art will recognize, with the appropriate dosage ultimately being at the discretion of the attendant physician. Dosage regimens may be adjusted to provide the optimum therapeutic response. A therapeutically effective amount is also one in which any toxic or detrimental effects of the components are outweighed by the therapeutically beneficial effects.

Preferably, the compounds of the present invention, are administered at a dosage and for a time such that the number of hot flushes is reduced as compared to the number of hot flushes prior to the start of treatment. Such treatment can also be beneficial to reduce the overall severity or intensity distribution of any hot flushes still experienced, as compared to the severity of hot flushes prior to the start of the treatment.

The term “hot flash” is an art-recognized term that refers to an episodic disturbance in body temperature typically consisting of a sudden skin flushing, usually with accompanied perspiration in a subject.

The phrases “vasomotor symptoms,” “vasomotor instability symptoms” and “vasomotor disturbances” include, but are not limited to, hot flushes (flashes), insomnia, sleep disturbances, mood disorders, irritability, excessive perspiration, night sweats, fatigue, and the like, caused by, inter alia, thermoregulatory dysfunction.

The terms “component”, “drug”, “pharmacologically active agent”, “agent”, and “medicament” are used interchangeably herein to refer to a compound or compounds, e.g., antibody, small molecule, nucleic acid molecule, peptide, oligopeptide, polypeptide, or protein, or compositions containing such, which when administered to an organism (human or animal) induces a desired pharmacologic and/or physiologic effect by local and/or systemic action. The component herein may contain NRI activity or NRI/SRI activity in combination with a 5-HT₂ₐ receptor antagonist activities.

The terms “synergy” and “synergistic” refer to instances where the effectiveness of a composition comprising two or more components, such as desipramine and MDL-100907, exceeds the sum of the efficacies of the individual components taken alone. Thus, using a synergistic compound combination may allow for use of a lower overall concentration of the compound or the realization of an enhanced hot flush alleviating effect at a comparable dosage.

The term “modulation” refers to the capacity to either enhance or inhibit a functional property of a biological activity or process, for example, receptor binding, signaling activity. Such enhancement or inhibition may be contingent on the occurrence of a specific event, such as activation of a signal transduction pathway and/or may be manifest only in particular cell types. The modulator is intended to comprise any compound, e.g., antibody, small molecule, peptide, oligopeptide, polypeptide, or protein, preferably small molecule or peptide.

The term “inhibit” refers to the act of diminishing, suppressing, alleviating, preventing, reducing or eliminating, whether partial or whole, a function or an activity. The term “inhibit” can be applied to both in vitro as well as in vivo systems. As used herein, the term “inhibitor” refers to any agent that inhibits.

The term “NRI/SRI” refer to a compound having dual activity as a serotonin reuptake inhibitor and as a norepinephrine reuptake inhibitor.

Within the present invention, the NRIs and the NRI/SRIs may be prepared in the form of pharmaceutically acceptable salts. As used herein, the term “pharmaceutically acceptable salts” refers to salts prepared from pharmaceutically acceptable non-toxic acids, including inorganic salts, and organic salts. Suitable non-organic salts include inorganic and organic acids such as acetic, benzenesulfonic, benzoic, camphorsulfonic, citric, ethenesulfonic, fumaric, gluconic, glutamic, hydrobromic, hydrochloric, isethionic, lactic, malic, maleic, mandelic, methanesulfonic, mucic, nitric, pamoic, pantothenic, phosphoric, succinic, sulfuric, tartaric acid, p-toluenesulfonic and the like. Particularly preferred are hydrochloric, hydrobromic, phosphoric, and sulfuric acids, and most preferably is the hydrochloride salt.

Examples of NRIs are maprotiline, desipramine, imipramine amoxapine, doxepin, bupropion, lofepramin, reboxetine, and amitriptyline. Examples of NRI/SRI compounds are venlafaxine, desvenlafaxine (DVS-233), milnacipran and duloxetine.

The compounds of the present invention may also comprise pharmaceutical acceptable adjuvants, carriers, and/or excipients, and the like which are well known in the art, for example as described in the Hand Book of Pharmaceutical Excipients, second edition, American Pharmaceutical Association, 1994 (incorporated herein by reference).

Further, the compounds of the present invention may exist in unsolvated as well as in solvated forms with pharmaceutically acceptable solvents such as water, ethanol, and the like. In general, the solvated forms are considered equivalent to the unsolvated forms for the purpose of the present invention.

Some of the compounds of the present invention may contain chiral centers and such compounds may exist in the form of isomers (i.e. enantiomers). The present invention includes all such isomers and any mixtures thereof including racemic mixtures.

The NRI activity in combination with 5-HT₂ₐ receptor antagonist activity may reside within the same chemical compound or in two or more different chemical compounds. Furthermore, dual NRI/SRI activity in combination with 5-HT₂ₐ receptor antagonist activity may reside within the same chemical compound or in two or more different chemical compounds. For example, a single compound may have NRI activity, and a 5-HT₂ₐ receptor antagonist activity, or a single compound may have NRI activity, SRI activity and a 5-HT₂ₐ receptor antagonist activity.

The NRI compound may be administered to patients in the daily dosage range of from about 0.1 to 500 mg per day. A more preferred range is about 10 to 300 mg per day, the most preferred daily dosage being about 100 to 200 mg per day. The dual acting NRI/SRI compound may be administered to patients in the daily dosage range of from about 0.10 to 200 mg per day. A more preferred range is about 1-100 mg per day, the most preferred daily dosage being about 10 to 50 mg per day.

A pharmaceutical for use in accordance with the present invention comprises NRI or NRI/SRI in combination with at least one 5-HT₂ₐ receptor antagonist, or a pharmaceutically acceptable salt thereof, together with a pharmaceutically acceptable carrier. The composition may comprise one or more NRI(s), or one or more each of NRI/SRI(s) as active ingredient(s) with one or more 5-HT₂ₐ receptor antagonist(s), together with one or more pharmaceutically acceptable carrier(s).

Such compositions are prepared in accordance with acceptable pharmaceutical procedures, such as described in Remington's Pharmaceutical Sciences, 17th edition, ed. Alfonoso R. Gennaro, Mack Publishing Company, Easton, Pa. (1985). Pharmaceutically acceptable carriers are those that are compatible with the other ingredients in the formulation and biologically acceptable.

The compounds of this invention may be administered orally or parenterally, neat or in combination with conventional pharmaceutical carriers. Applicable solid carriers can include one or more substances that may also act as flavoring agents, lubricants, solubilizers, suspending agents, fillers, glidants, compression aids, binders or tablet-disintegrating agents or an encapsulating material. In powders, the carrier is a finely divided solid that is in admixture with the finely divided active ingredient. In tablets, the active ingredient is mixed with a carrier having the necessary compression properties in suitable proportions and compacted in the shape and size desired. The powders and tablets preferably contain up to 99% of the active ingredient. Suitable solid carriers include, for example, calcium phosphate, magnesium stearate, talc, sugars, lactose, dextrin, starch, gelatin, cellulose, methyl cellulose, sodium carboxymethyl cellulose, polyvinylpyrrolidine, low melting waxes and ion exchange resins.

Liquid carriers may be used in preparing solutions, suspensions, emulsions, syrups, and elixirs. The active ingredient of this invention can be dissolved or suspended in a pharmaceutically acceptable liquid carrier such as water, an organic solvent, a mixture of both or pharmaceutically acceptable oils or fat. The liquid carrier can contain other suitable pharmaceutical additives such as solubilizers, emulsifiers, buffers, preservatives, sweeteners, flavoring agents, suspending agents, thickening agents, colors, viscosity regulators, stabilizers, or osmo-regulators. Suitable examples of liquid carriers for oral and parenteral administration include water (particularly containing additives as above, e.g. cellulose derivatives, preferably sodium carboxymethyl cellulose solution), alcohols (including monohydric alcohols and polyhydric alcohols e.g. glycols) and their derivatives, and oils (e.g. fractionated coconut oil and arachis oil). For parenteral administration the carrier can also be an oily ester such as ethyl oleate and isopropyl myristate. Sterile liquid carriers are used in sterile liquid form compositions for parenteral administration.

Liquid pharmaceutical compositions, which are sterile solutions or suspensions, can be administered by, for example, intramuscular, intraperitoneal or subcutaneous injection. Sterile solutions can also be administered intravenously. Oral administration may be either liquid or solid composition form.

Preferably the pharmaceutical composition is in unit dosage form, e.g. as tablets, capsules, powders, solutions, suspensions, emulsions, granules, or suppositories. In such form, the composition is sub-divided in unit dose containing appropriate quantities of the active ingredient; the unit dosage forms can be packaged compositions, for example packeted powders, vials, ampoules, prefilled syringes or sachets containing liquids. The unit dosage form can be, for example, a capsule or tablet itself, or it can be the appropriate number of any such compositions in package form.

Generally, the NRI or NRI/SRI or a pharmaceutically acceptable salt thereof will be present at a level of from about 0.1%, by weight, to about 90% by weight, based on the total weight of the pharmaceutical composition, and SRI or a pharmaceutically acceptable salt thereof, if present, will be present at a level of from about 0.1%, by weight, to about 90% by weight, based on the total weight of the pharmaceutical composition. Preferably, the NRI or a pharmaceutically acceptable salt thereof will be present at a level of at least about 1%, by weight, and the SRI, if present, will be present at a level of at least about 1%, based on the total weight of the pharmaceutical composition. More preferably, the NRI or NRI/SRI or a pharmaceutically acceptable salt thereof will be present at a level of at least about 5%, by weight, and the SRI, if present, will be present at a level of at least about 5%, based on the total weight of the pharmaceutical composition. Even more preferably, the NRI or NRI/SRI or a pharmaceutically acceptable salt thereof will be present at a level of at least about 10%, by weight, and the SRI, if present, will be present at a level of at least about 10%, based on the total weight of the pharmaceutical composition. Yet even more preferably, the NRI or NRI/SRI or a pharmaceutically acceptable salt thereof will be present at a level of at least about 25%, by weight, and the serotonin reuptake inhibitor will be present at a level of at least about 25%, based on the total weight of the pharmaceutical composition.

The term “combination therapy” refers to the administration of two or more therapeutic agents or compounds to treat a therapeutic condition or disorder described in the present disclosure, for example hot flush, sweating, thermoregulatory -related condition or disorder, or other. Such administration includes co-administration of these therapeutic agents or compounds in a simultaneous manner, such as in a single compound having NRI, NRI/SRI or 5-HT₂ₐ activity or in multiple, separate compounds for each NRI, NRI/SRI or 5-HT₂ₐ activities. In addition, such administration also includes use of each type of therapeutic agent in a concurrent manner. In either case, the treatment regimen will provide beneficial effects of the drug combination in treating the conditions or disorders described herein.

The term “central nervous system” or “CNS” includes the brain and the spinal cord. The term “peripheral nervous system” or “PNS” includes all parts of the nervous system that are not part of the CNS, such as cranial and spinal nerves and the autonomic nervous system.

The route of administration may be any route, which effectively transports the NRI(s) and/or NRI/SRI(s), and 5-HT₂ₐ receptor antagonist(s) to the appropriate or desired site of action, such as oral, nasal, pulmonary, transdermal, such as passive or iontophoretic delivery, or parenteral, e.g. rectal, depot, subcutaneous, intravenous, intraurethral, intramuscular, intranasal, ophthalmic solution or an ointment. Furthermore, the administration of NRI(s) or NRI/SRI(s) with 5-HT₂ₐ receptor antagonist(s) may be concurrent, separate, simultaneous, or staggered in time.

The term “subject” or “patient” refers to a mammal including the human species that is treatable with the compositions, and/or methods of the present invention. The term “subject” or “subjects” is intended to refer to both the male and female gender unless one gender is specifically indicated. Accordingly, the term “patient” comprises any mammal which may benefit from treatment or prevention of vasomotor symptoms, such as a human, especially if the mammal is female, either in the pre-, peri- or post- menopausal period. Furthermore, the term patient comprises female mammals including humans and, among humans, not only women of advanced age who have passed through menopause but also women who have undergone hysterectomy or for some other reason have suppressed estrogen production, such as those who have undergone long-term administration of corticosteroids, suffer from Cushing's syndrome or have gonadal dysgenesis.

The term “premature menopause” or “artificial menopause” refers to ovarian failure of unknown cause that may occur before age 40. It may be associated with smoking, living at high altitude, or poor nutritional status. Artificial menopause may result from oophorectomy, chemotherapy, radiation of the pelvis, or any process that impairs ovarian blood supply.

The term premenopausal means before the menopause, the term perimenopausal means during the menopause and the term postmenopausal means after the menopause.

Ovariectomy means removal of an ovary or ovaries and can be effected according to Merchenthaler et al. Maturitas, Nov. 16, 1998; 30(3): 307-316.

### EXAMPLES

The present invention is further defined in the following Examples, in which all parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions.

GENERAL METHODS

Reagents: MDL-100907 (5-HT₂ₐ receptor antagonist) was synthesized as described in WO91/18602. Desipramine was prepared as described in U.S. Pat. No. 3,454,554. Venlafaxine was prepared as described in U.S. Pat. No 4,535,186. The following reagents were purchased commercially: DOI (Sigma),morphine alkaloid pellets (Murty Pharmaceuticals, Lexington, Ky.), ketamine (Phoenix Pharmaceuticals, Belmont, Calif.) and naloxone (Research Biochemicals International, St. Louis, Mo.).

Dosing: All doses were prepared based on mg/kg. All test compounds except MDL-100907 were dissolved in sterile water. MDL-100907 was dissolved in Tween 80. All drugs were injected subcutaneously (sc), and used at the following dosages: DOI (0.3 mg/kg), venlafaxine (10 mg/kg), desipramine (1.0 mg/kg), and MDL-100907 (0.01 and 0.1 mg/kg). Ketamine (Ketaject, Phoenix Pharmaceuticals, Belmont, Calif.) was injected intramuscularly at a dosage (40 mg/kg) that was determined to be mildly sedative but did not cause a change in tail skin temperature.

Animals: Ovariectomized Sprague-Dawley rats (180-220 g) were obtained from a commercial vendor (Taconic, Germantown, N.Y.) and individually housed under 12 h light/dark cycle. Animals were provided with standard rat chow and water ad libitum. Animals were housed in a room maintained at 25° C., but were treated and tested in a room maintained at 21° C.

Morphine-dependent model: Ovariectomized rats were injected once daily for 8-9 days with vehicle to minimize stress responses and then administered compound(s) on test day. On day 4 of dosing, morphine dependence was induced by sc implantation of two slow-release morphine pellets (75 mg/pellet) in the dorsal scapular region. This model is based upon an established morphine-dependent naloxone-induced flush (MD model) paradigm that is reversible by estrogen treatment (Katovich et al., Proceedings of the Society for Experimental Biology & Medicine, 1990. 193(2): p.129-35). Four to six days after implantation, morphine withdrawal was induced with an opioid antagonist (naloxone) that causes a transient increase in TST. In a typical experiment, rats were administered their final dose of test compound 1 h prior to naloxone injection. Rats were mildly sedated with ketamine and a thermistor connected to a MacLab data acquisition system was taped to the base of the tail. Tail skin temperature was then monitored continuously for 35 minutes to establish a baseline temperature. Naloxone was subsequently administered and TST was measured for an additional 60 min (total recording time 95 min).

Statistical analysis: To analyze changes in TST induced by naloxone in morphine-dependent rats, all data were analyzed using a two factors repeated measure. The factors were “treatment” and 37 time” (repeated). The model was fit to test whether there were significant differences in the responses between treatment groups. The data were analyzed at 5 minute intervals from 20 minutes (−20) prior to the naloxone administration (referred to as time 0) to 60 minutes after the treatment. Multiple comparisons (LSD p-values) among the treatment groups at each time point were used for the analysis, however the changes in TST is greatest at 15 minutes post-naloxone administration and this time point provides the best indicator of flush abatement. The ability of compound to abate the MDL-100907-induced flush was analyzed at 15 min pre-naloxone (−15 min ) using the same analysis as that used for the significance determination of post-naloxone administration. When appropriate estimation of the ED₅₀ value was calculated. The ED₅₀ value was determined using a log scale and the line was fit between the maximal (15 min post naloxone ΔTST) and minimal response (average baseline temperature prior to naloxone). The ED₅₀ value is reported as the dose of test compound that abates 50% of the naloxone-induced flush.

### Example 1

Effect of a 5-HT₂ₐ receptor agonist on a 5-HT₂ₐ Receptor Antagonist Induced Flush in Pre-clinical Models of Vasomotor Instability

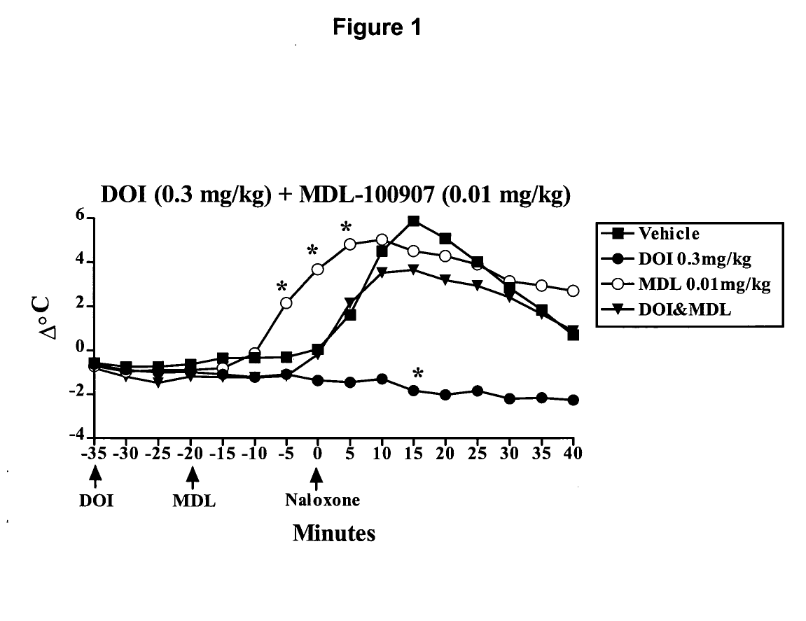

Method used as described in the general method section under morphine-dependent rat model with the following exceptions: Rats were injected subcutaneously with vehicle (sterile H20) or DOI (5-HT₂ₐ agonist, Sigma; sterile H20) and administered at 0.3 mg/kg, MDL-100907 (5-HT₂ₐ receptor antagonist) was synthesized as described in WO9118602, dissolved in Tween 80 and administered at 0.01 mg/kg. DOI was administered 15 min prior to MDL-100907. Changes in TST (Δ° C., Mean) over time in the morphine-dependent rat model depict DOI abatement the 5-HT₂ₐ receptor antagonist-induced flush. At maximal flush (15 min post-naloxone; Δ° C., Mean+SEM), MDL-100907 reversed the DOI alleviation of the naloxone-induced flush (FIG. 1). These results indicate that the 5-HT₂ₐ receptor system is involved in thermoregulation.

### Example 2

Effect of Compounds with NRI and 5-HT₂ₐ Receptor Antagonist Activity in Alleviating Vasomotor Instability

Methods are described in the general method section under morphine-dependent rat model with the following exceptions: Rats were injected subcutaneously with vehicle (sterile H20), desipramine (which was prepared as described in U.S. Pat. No. 3,454,554, dissolved in sterile H20 and administered at 1.0 mg/kg), MDL-1 00907 (was synthesized as described in WO91/18602, dissolved in Tween 80 at 0.01 mg/kg) or a combination of MDL-100907 and desipramine. MDL-100907 (0.01) was administered 55 min and desipramine was administered 40 min prior to naloxone injection.

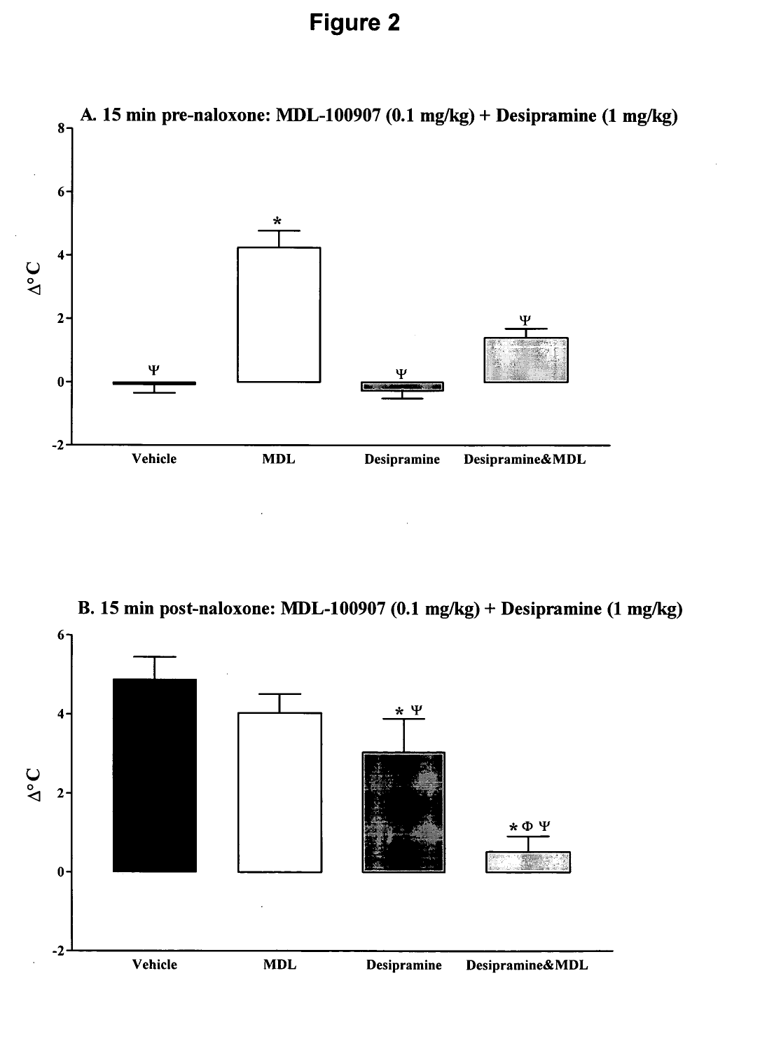

Changes in TST (Δ° C., Mean) over time in the morphine-dependent rat model demonstrated that MDL-100907 induced a significant increase in TST prior to administration of naloxone and enhanced the naloxone-induced hot flush. Desipramine reversed the 5-HT₂ₐ receptor antagonist-induced pre-naloxone flush (FIG. 2A). This elevated TST induced by MDL-100907 inferred that the 5-HT₂ₐ receptor is involved in vasomotor instability. Furthermore, these data indicated that desipramine's efficacy can be enhanced during a naloxone-induced flush if the 5-HT₂ₐ receptor is blocked by MDL-100907 (FIG. 2B).

### Example 3

Effect of Compounds with Dual NRI/SRI Activity and a 5-HT₂ₐ Receptor Antagonist Activity in Alleviating Vasomotor Instability

Method as described in the general method section with the following exceptions: Rats were injected subcutaneously with vehicle (sterile H20), venlafaxine (dissolved in sterile H20 and administered at 10 mg/kg), MDL-100907 (Sigma, dissolved in Tween 80 at 0.01 mg/kg) or with a combination of venlafaxine and MDL-100907. Venlafaxine was synthesized as described in U.S. Pat. No. 4535186. MDL-100907 (0.01) was administered 55 min and venlafaxine was administered 40 min prior to naloxone injection.

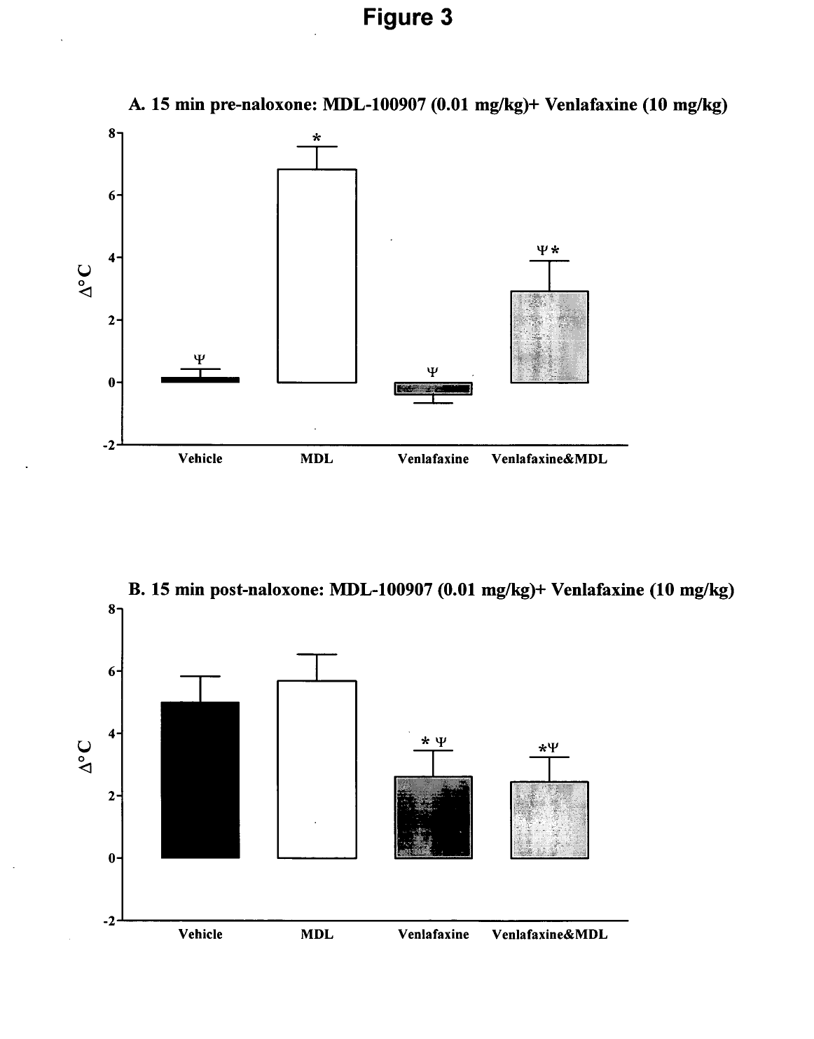

Changes in TST (Δ° C., Mean) over time in the morphine-dependent rat model demonstrated that MDL-100907 induced a significant increase in TST prior to administration of naloxone and enhanced the naloxone-induced hot flush. Venlafaxine reversed the 5-HT₂ₐ receptor antagonist-induced pre-naloxone flush (FIG. 3A). This elevated TST induced by MDL-100907 inferred that the 5-HT₂ₐ receptor is involved in vasomotor instability. Furthermore, these data indicated that venlafaxine's efficacy is in part mediated by an increase in norepinephrine signaling and blocking the 5-HT₂ₐ receptor system can enhance this effect (FIG. 3B).

When ranges are used herein for physical properties, such as molecular weight, or chemical properties, such as chemical formulae, all combinations and subcombinations of ranges specific embodiments therein are intended to be included.

The disclosures of each patent, patent application and publication cited or described in this document are hereby incorporated herein by reference, in its entirety.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

What is claimed is:

1. A method for treating or preventing vasomotor symptoms in a subject, comprising the step of:
administering to said subject a therapeutically effective amount of one or more compounds having norepinephrine reuptake inhibitor activity and 5-HT₂ₐ antagonist activity.

2. The method of claim 1, wherein the norepinephrine reuptake inhibitor activity and 5-HT₂ₐ receptor antagonist activity are provided by a single compound.

3. The method of claim 1, wherein the compounds are administered as a combination therapy comprising administering to said subject an effective amount of a first component which is a norepinephrine reuptake inhibitor, its derivatives and or pharmaceutically acceptable salts thereof in combination with an effective amount of a second component which is a 5-HT₂ₐ receptor antagonist, its derivatives and or pharmaceutically acceptable salts thereof.

4. The method of claim 3, wherein the first component is selected from the group consisting of maprotiline, reboxetine, desipramine, imipramine amoxapine, doxepin, bupropion, lofepramin, amitriptyline, and combinations and pharmaceutically acceptable salts thereof.

5. The method of claim 3, wherein the second component is (+)-α-(2,3-dimethoxy phenyl)-1-[2-(4-fluorophenyl)ethyl]-4-piperidinemethanol or pharmaceutically acceptable salt thereof.

6. The method of claim 3, wherein:
a. the first component is selected from the group consisting of maprotiline, reboxetine, desipramine, nisoxetine, imipramine, amoxapine, doxepin, lofepramin and amitriptyline and pharmaceutically acceptable salt thereof; and
b. the second component is (+)-α-(2,3-dimethoxyphenyl)-1-[2-(4-fluorophenyl)ethyl]-4-piperidinemethanol or a pharmaceutically acceptable salt thereof.

7. A method for treating or preventing vasomotor symptoms in a subject, comprising the step of:
administering to said subject a therapeutically effective amount of one or more compounds having NRI/SRI activity, and 5-HT₂ₐ antagonist activity.

8. The method of claim 7, wherein the NRI/SRI activity and 5-HT₂ₐ receptor antagonist activity are provided by a single compound.

9. The method of claim 7, wherein the compounds are administered as a combination therapy comprising administering to said subject an effective amount of a first component which is a NRI/SRI, its derivatives and or pharmaceutically acceptable salts thereof in combination with an effective amount of a second component which is a 5-HT₂ₐ receptor antagonist, its derivatives and or pharmaceutically acceptable salts thereof.

10. The method of claim 9, wherein the first component is selected from the group consisting of venlafaxine, desvenlafaxine (DVS-233), milnacipran, duloxetine, and combinations and pharmaceutically acceptable salts thereof.

11. The method of claim 9, wherein the second component is (+)-α-(2,3-dimethoxy phenyl)-1-[2-(4-fluorophenyl)ethyl]-4-piperidinemethanol or a pharmaceutically acceptable salt thereof.

12. The method of claim 9, wherein:
a. the first component is selected from the group consisting of venlafaxine, desvenlafaxine (DVS-233), milnacipran and duloxetine and pharmaceutically acceptable salts thereof; and
b. the second component is (+)-α-(2,3-dimethoxy phenyl)-1-[2-(4-fluorophenyl)ethyl]-4-piperidinemethanol or a pharmaceutically acceptable salt thereof.

13. The method of any of claims 1-12, wherein the vasomotor symptom is hot flush.

14. The method of any of claims 1-12, wherein the subject is human.

15. The method of claim 14, wherein the human is female patient.

16. The method of claim 15, wherein the female patient is perimenopausal.

17. The method of claim 15, wherein the female patient is menopausal.

18. The method of claim 15, wherein the female patient is post-menopausal.

19. The method of claim 14, wherein the subject is male patient.

20. The method of claim 19, wherein the male patient is naturally, chemically or surgically andropausal.

21. The method of claim 4 or 9, wherein the administration of norepinephrine reuptake inhibitor and 5-HT₂ₐ antagonist is concurrent.

22. The method of claim 4 or 9, wherein the administration of norepinephrine reuptake inhibitor and 5-HT₂ₐ antagonist is simultaneous.

23. A pharmaceutical formulation comprising a norepinephrine reuptake inhibitor and a 5-HT₂ₐ antagonist in a pharmaceutically acceptable excipient.

24. A pharmaceutical formulation comprising a NRI/SRI and a 5-HT₂ₐ antagonist in a pharmaceutically acceptable excipient.
